(19) United States
(12) Patent Application Publication
(10) Pub. No.: US 2020/0132583 A1
(43) Pub. Date: Apr. 30, 2020

(54) SIGNAL AMPLIFICATION IN BIOSENSOR DEVICE

(71) Applicant: AVIANA MOLECULAR TECHNOLOGIES, LLC, Orlando, FL (US)

(72) Inventors: Soumen Das, Orlando, FL (US); John Martin Hamlyn, Columbia, MD (US)

(73) Assignee: AVIANA MOLECULAR TECHNOLOGIES, LLC, Orlando, FL (US)

(21) Appl. No.: 16/629,934

(22) PCT Filed: Jul. 6, 2018
(86) PCT No.: PCT/US18/40977

§ 371 Date: Jan. 9, 2020

Related U.S. Application Data
(60) Provisional application No. 62/531,238, filed on Jul. 11, 2017.

(51) Int. Cl. G01N 15/06; G01N 33/553

(52) U.S. Cl. CPC G01N 15/0606; G01N 2015/0065; G01N 33/553

(57) ABSTRACT

An acoustic wave biosensor component is provided. A method of amplifying the biosensor signal is also provided, including applying a polymer or metallic material to the analyte after the analyte is attached to the capture agent on the biosensor.

### CROSS-REFERENCE TO RELATED APPLICATION

This application claims the benefit of priority of U.S. Provisional Patent Application No. 62/531,238, filed Jul. 11, 2017, the entire contents of which are hereby incorporated by reference.

### FIELD

The present disclosure relates generally to devices and methods for analyzing test samples containing target analytes including proteins, cells and nucleic acids. More particularly, the disclosure relates to modulating or amplifying signals from analyte bindings, and the platform technology disclosed herein is suitable for the development of a variety of biosensors with high sensitivity and selectivity.

### BACKGROUND

Acoustic wave sensors use a detection arrangement that is based on detecting perturbations (e.g., changes in amplitude, frequency, phase, time delay, etc.) to mechanical or acoustic waves traveling through materials sensitive to such perturbations. As an acoustic wave propagates through or on the surface of an acoustic wave sensor material, any changes to the physical or chemical characteristics of the sensor material (including, e.g. mass of the analyte and/or viscosity changes on the wave path, etc.) may affect the velocity and/or amplitude of the surface acoustic or bulk acoustic waves. These changes may be detected and correlated to the corresponding quantities laid on their surface, and then measured to sense/detect the physical/chemical characteristics of analytes located in/on the sensor material. Unfortunately, the binding between the target molecule and the sensor surface may be weak, and the prior art acoustic wave sensors often lack sensitivity and do not operate efficiently when they are presented with the target. Therefore, there is a need in the art for devices and methods that increase acoustic wave sensor signal detection sensitivity and selectivity.

### SUMMARY OF THE INVENTION

In one aspect, the disclosure provides method of amplifying signal in a biosensor, including the steps of: applying a sample to the biosensor having a capture reagent, wherein the capture reagent comprises one or more first recognition moieties for binding an analyte, and wherein the capture reagent is immobilized on the biosensor; and introducing a signal amplifying material, wherein the signal amplifying material has one or more second recognition moieties for binding to the analyte.

In an embodiment, the signal amplifying material is a polymer, metal, or metal oxide.

In an embodiment, the signal amplifying material is polystyrene.

In an embodiment, the signal amplifying material is in the form of a bead of various materials.

In an embodiment, the bead has an average diameter in the range of about 1 nm to about 100 μm.

In an embodiment, the signal amplifying material is introduced after the analyte binds to the biosensor.

In an embodiment, the signal amplifying material is introduced prior to the analyte binding to the biosensor.

In an embodiment, the method includes measuring a base level signal prior to applying the sample to the biosensor.

In an embodiment, the method includes measuring a test level signal after the binding of the signal amplifying material to the analyte.

In an embodiment, the method includes comparing the base level signal to the test level signal to determine the presence of the analyte in the sample.

In an embodiment, the first recognition moiety is a moiety for binding to whole cells, bacteria, eukaryotic cell, tumor cell, virus, fungus, parasite, spore, nucleic acid, small molecule, or protein.

In an embodiment, the first recognition moiety is selected from the group consisting of antibody, antibody fragment, single domain antibody, affirmer and aptamer.

In an embodiment, the second recognition moiety is a moiety for binding to whole cells, bacteria, eukaryotic cell, tumor cell, virus, fungus, parasite, spore, nucleic acid, or protein.

In an embodiment, the second recognition moiety is selected from the group consisting of antibody, antibody fragment, single domain antibody, affirmer and aptamer conjugated with polymer, metal or metal oxide material.

In an embodiment, the sample is an environmental or biological sample.

In an embodiment, the biological sample is blood, serum, plasma, urine, sputum, fecal matter, nasal or vaginal swab, tears, cerebrospinal fluid, pericardial fluid, intraocular fluid, cyst fluid or saliva.

In one aspect, the disclosure provides a method for determining the presence or quantity of an analyte in a sample the method including the steps of: applying a sample to the biosensor having a capture reagent having one or more first recognition sites for binding an analyte, wherein the capture reagent is immobilized on the biosensor; introducing a signal amplifying material, wherein the polymer or metallic material has one or more second recognition sites to bind the analyte; and measuring any change in amplitude, phase or frequency of biosensor signal as a result of analyte binding to the signal amplifying material.

In one aspect, the disclosure provides a biosensor component including: a piezoelectric substrate; a capture reagent, wherein the capture reagent is immobilized on the piezoelectric substrate and wherein the capture agent has a first recognition site for an analyte, and a signal amplifying material having a second recognition site for the analyte.

In an embodiment, the biosensor further includes an anchor substance to attach the capture reagent to the piezoelectric substrate.

In an embodiment, the piezoelectric substrate is selected from the group consisting of aluminum (Al), lithium niobate (LiNbO3), lithium tantalate (LiTaO3), silicon dioxide (SiO2), and borosilicate.

In an embodiment, the anchor substance binds to the surface of the piezoelectric substrate through a silane group or a thiol group.

In an embodiment, the anchor substance comprises a linker protein selected from avidin, oligonucleotide, or polynucleotide.

In an embodiment, the linker protein is avidin selected from the group consisting of neutravidin, natural avidin, streptavidin, and any combination thereof.

In an embodiment, the capture reagent comprises a biotin moiety for binding to the linker protein of the anchor substance.

In an embodiment, the first recognition site is configured to bind whole cells, bacteria, eukaryotic cell, tumor cell, virus, fungus, parasite, spore, nucleic acid, or protein.

In an embodiment, the biosensor component further includes an acoustic wave transducer.

In an embodiment, the acoustic wave transducer generates bulk acoustic waves.

In an embodiment, the bulk acoustic wave is selected from the group consisting of thickness shear mode, acoustic plate mode, and horizontal plate mode.

In an embodiment, the biosensor component is a film bulk acoustic-wave resonator-based (FBAR-based) device.

In an embodiment, the acoustic wave transducer generates surface acoustic waves.

In an embodiment, the surface acoustic wave is selected from the group consisting of shear horizontal surface acoustic wave, surface traverse wave, Rayleigh wave, and Love wave.

In one aspect, the disclosure provides a bulk wave resonator comprising the biosensor component of any one of the foregoing.

Some embodiments relate to a method of amplifying signal to the biosensor, comprising: applying a sample to the biosensor having a capture reagent, wherein the capture reagent comprises one or more first recognition moieties for binding an analyte, and wherein the capture reagent is immobilized on the biosensor; and introducing a signal amplifying material, wherein the signal amplifying material has one or more second recognition moieties for binding to the analyte.

Some embodiments relate to a method for determining the presence or quantity of an analyte, the method comprising: applying a sample to the biosensor having a capture reagent having one or more first recognition sites for binding an analyte, wherein the capture reagent is immobilized on the biosensor; introducing a signal amplifying material, wherein the polymer or metallic material has one or more second recognition sites to bind the analyte in a different portion of the analyte; and measuring any change in amplitude, phase or frequency of biosensor signal as a result of analyte binding to the signal amplifying material.

Some embodiments relate to a biosensor component comprising: a piezoelectric substrate; a capture reagent, wherein the capture reagent is immobilized on the piezoelectric substrate and wherein the capture agent has a first recognition site for an analyte, and a signal amplifying material having a second recognition site for the analyte.

Some embodiments relate to a bulk wave resonator comprising the biosensor component described herein.

### Certain Terminology

The following terms shall have the meaning ascribed to them below.

“Anchor substance” denotes a coating material that binds both to (i) the piezoelectric substrate (for “direct” binding) or metal part of the sensor surface or to an intermediary coating thereon and (ii) to a “capture reagent” (as defined below). The term includes avidins, a member of a family of proteins functionally defined by their ability to bind biotins, which serve as their specific binding partners (i.e. avidin, streptavidin, neutravidin), as well as oligo and polynucleotides and proteins having a specific affinity binding partner which could be used to modify a capture reagent and therefore to cause the capture reagent to bind to the anchor-coated piezoelectric/sensor material. Also included are naturally occurring carbohydrate-binding lectins, which bind to carbohydrate groups e.g., on antibodies and antibody fragments (Fe fragments) and single domain antibody and nucleotide fragments such as aptamers. Generally, it is not preferred to use a capture reagent as an anchor because of the risk of changing the conformation or even partially denaturing the capture reagent which would affect accuracy of the test. Oligo and polynucleotides can bind to piezoelectric materials through ionic or dipole sites, either directly or through intermediary silver coating applied by ion exchange methods. Their specific binding partners are complementary nucleotide molecule and those can be used to modify capture reagents.

“Capture reagent” means a substance that specifically binds to an analyte in a biological sample, such that it can be used to identify and/or quantitate the analyte by capturing it from the biological sample. The term includes antibodies, aptamers and antibody fragments thereof without limitation. A capture reagent will bind to the anchor substance with or without modification with a linking group which is a specific binding partner for the anchor substance (e.g., biotinylation or complementary nucleic acid). In other words, the capture reagent is or comprises a specific binding partner for the anchor substance and simultaneously specifically recognizes an analyte.

“Direct” or “directly” as applied to binding of an anchor substance to a substrate surface means binding to the substrate surface without application of an intermediary coating thereon. The substrate surface may be modified, for example by application of plasma, ultraviolet radiation, or by ion exchange deposition of silver ions, which replace metal ions on the surface but do not deposit an additional layer of intermediary material on the surface metal ions on the piezoelectric surface.

A “small organic molecule” refers to an organic molecule, either naturally occurring or synthetic, that has a molecular weight of more than about 10 daltons and less than about 2500 daltons, preferably less than about 2000 daltons, preferably between about 10 to about 1000 daltons, more preferably between about 10 to about 500 daltons.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

### BRIEF DESCRIPTION OF THE DRAWINGS

FIGS. 1A-1D illustrate the response of a thiolated-neutravidin-decorated Surface Acoustic Wave sensor (SAW) to the binding of biotinylated beads: FIG. 1A illustrates the binding scheme of the thiolated-neutravidin-decorated surface to the biotinylated polystyrene beads; FIG. 1B shows the mass-evoked frequency shifts induced by the binding of polystyrene beads to a SAW sensor; FIG. 1C shows the fluorescence microscopy after washing of the device to remove excess biotinylated polystyrene beads; and FIG. 1D shows the frequency shifts with different bead dilutions.

FIG. 2 shows an exemplified affinity based/nanoparticle strategy for the capture and enhanced detection of analytes by mass amplification on the aluminum surface of a SAW device.

### DETAILED DESCRIPTION

The present disclosure is based, at least in part, on the discovery that signal amplifying material (e.g., polypeptides, proteins, protein complexes, beads, polystyrene beads, and the like) may be directly bound to or conjugated to a target analyte or secondary antibody (e.g. an antibody, antibody fragments, polynucleotides, polypeptides with binding activity, and the like) that has been applied to a biosensor surface. The signal amplifying material may be directly bound to or conjugated to the target analyte, or may be bound to or conjugated to a second layer of signal amplifying material (e.g., to create stacked layers of amplifying material). The signal amplifying materials and techniques herein provide significant advantages over prior art methods by increasing both signal sensitivity and selectivity by increasing mass loading or viscosity change in SAW sensor.

### Biosensor Component

Some embodiments relate to a biosensor component comprising a piezoelectric substrate; a capture reagent, wherein the capture reagent is immobilized on the piezoelectric substrate and wherein the capture agent has a first recognition site for an analyte, and a signal amplifying material having a second recognition site for the analyte.

The surface of the sensor can be a metal layer (e.g., aluminum or aluminum alloy, gold, silver, titanium, chromium, platinum, tungsten, etc.) or have no metal surface, deposited on a piezoelectric crystal material. In some embodiments, the surface of the sensor can be a metal layer (e.g., aluminum or aluminum alloy), deposited on a piezoelectric crystal material. In some embodiments, sections of the sensor may contain the metal coating alternating with crystal or may be covered with dielectric material layer. In some embodiments, the dielectric layer can be a polymer or ceramic layer. In some embodiments, the dielectric layer can comprise SiO₂, poly(methyl methacrylate) (PMMA), zinc oxide, or aluminum nitrogen. In some embodiments, the dielectric layer can consist of metals such as gold, titanium, platinum, etc. In some embodiments, suitable crystals can be used along with various crystal cuts. In some embodiments, sections of the sensor may include a dielectric layer deposited on the piezoelectric substrate. In some embodiments, sections of the sensor may include a dielectric layer deposited on a metal layer, which in turn is deposited on the piezoelectric substrate. In some embodiments, sections of the sensor may include a metal layer deposited on a dielectric layer, which in turn is deposited on a metal layer. In some embodiments, sections of the sensor may include a first metal layer deposited on a dielectric layer, which is then deposited on a second metal layer, and the second metal layer is then deposited on the piezoelectric substrate. All suitable approaches regarding the use of SAW and Bulk Acoustic Wave (“BAW”) sensors for the detection of target analytes and can be based on the ability to decorate the sensor surface with a suitable coating described herein. For the detection of biomolecules, the sensor surface can be immobilized or modified with a suitable material that can selectively capture the desired target analyte.

The piezoelectric surface can be made of any suitable piezoelectric material. In some embodiments, the piezoelectric substrate is selected from the group consisting of quartz, lithium niobate and tantalate, 36° YX quartz, 36° YX lithium tantalate, langasite, langatate, langanite, lead zirconate titanate, cadmium sulfide, berlinite, lithium iodate, lithium tetraborate, bismuth germanium oxide, Zinc oxide, aluminium nitride, and gallium nitride. In some embodiments, the piezoelectric substrate is coated with a metal material selected from the group consisting of aluminum (Al), gold (Au), Al alloy and any combination thereof. In some embodiments, the piezoelectric substrate is coated with aluminum. In some embodiments, the piezoelectric substrate is coated with Al alloy. In some embodiments, the metal is selected from the group consisting of aluminum (Al), gold (Au), Aluminum alloy, silver, titanium, chromium, platinum, tungsten, and any combination thereof. In some embodiments, the dielectric layer can be a polymer or ceramic layer. In some embodiments, the dielectric layer can comprise SiO2, poly(methyl methacrylate) (PMMA), zinc oxide, or aluminum nitrogen.

In some embodiments, the biosensor component described herein further includes an anchor substance to attach the capture reagent to the piezoelectric substrate. In some embodiments, the anchor substance binds to the surface of the piezoelectric substrate through a silane group or a thiol group.

In some embodiments, the linker protein is avidin, Oligonucleotide, or polynucleotide.

In some embodiments, the linker protein is avidin selected from the group consisting of neutravidin, natural avidin, streptavidin, and any combination thereof.

The capture reagent can be an antibody or aptamer or other specific ligand or receptor formed from any of the following; biotinylated oligonucleotides, nucleotides, nucleic acids, (Pon, Richard T. (1991). “A long chain biotin phosphoramidate reagent for the automated synthesis of 5′-biotinylated oligonucleotides”. Tetrahedron Letters 32 (14): 1715-1718), proteins, peptides, and antibodies including IgA, IgG, IgM, IgE, enzymes, enzyme co-factors, enzyme inhibitors, membrane receptors, kinases, Protein A, Poly U, Poly A, Poly Lysine receptors, polysaccharides, chelating agents, carbohydrate and sugars.

In some embodiments, the capture reagent comprises a biotin moiety for binding to the binding protein of the anchor substance.

In some embodiments, the capture reagent comprises a moiety for binding to whole cells, bacteria, eukaryotic cell, tumor cell, virus, fungus, parasite, spore, nucleic acid, antibody, protein or small molecules. In some embodiments, the moiety is selected from the group consisting of antibody, protein fragments, peptides, polypeptides, affimer, antibody fragments, single domain antibody, aptamers or nucleotides. In some embodiments, the capture reagents can be an antibody. In some embodiments, the capture reagents can be an affimer or aptamer or chelating agent.

In some embodiments, surface modification includes a binding component having one or more functional group(s) used to immobilize the capturing agent. In some embodiments, the surface modification has one or more functional group(s) selected from the group consisting of N-Hydroxysuccinimide (NHS), sulfo-NHS, epoxy, carboxylic acid, carbonyl, maleimide and amine.

In some embodiments, the analyte can be a fragment of the Zika virus. In some embodiments, the analyte can be an E protein of the Zika virus.

Some of the exemplified detection methods are illustrated as a surface having an antibody attached as a capturing molecule. However, the method may not be limited to antibodies and can be adapted to immobilize other capture agents including but not limited to protein fragments, affimer, antibody fragments, aptamers or nucleotides on the sensor surface.

In some embodiments, the second recognition site is configured to bind whole cells, bacteria, eukaryotic cell, tumor cell, virus, fungus, parasite, spore, nucleic acid, small molecule, or protein.

In some embodiments, the biosensor component described herein further comprises an acoustic wave transducer. In some embodiments, the acoustic wave transducer generates BAWs.

In some embodiments, the bulk acoustic wave is selected from the group consisting of thickness shear mode, acoustic plate mode, and horizontal plate mode.

In some embodiments, the biosensor component is a film bulk acoustic-wave resonator-based (FBAR-based) device.

In some embodiments, the acoustic wave transducer generates surface acoustic waves.

In some embodiments, the SAW is selected from the group consisting of shear horizontal surface acoustic wave, surface traverse wave, Rayleigh wave, and Love wave.

### Bulk Acoustic Wave Resonator

Bulk Acoustic Wave (BAW) resonator is a device composed at least of one piezoelectric material sandwiched between two electrodes. The electrodes apply an alternating electric field to the piezoelectric material that creates stress resulting in the generation of BAWs. Some designs add one or more layers with high and low acoustic impedance to create a Bragg reflector or may suspend these layers. The BAW resonator can include several layers such as piezoelectric substrate (AlN, PZT, Quartz, LiNbO3, Langasite, etc.), electrodes (gold, aluminum, copper, etc.), Bragg reflector (high or low acoustic impedance material), layers that catch the analyte (bio-active layer, antibodies, antigen, gas sensitive layer, palladium, etc.) and any material which can propagate an acoustic wave. The BAW sensor can be a mix of the various layers described herein. The sensitive layer (layer to catch the analyte) can be in contact directly with the electrodes (A), or can be on the Bragg reflector, or can be on any material which can propagate an acoustic wave.

Some embodiments relate to a BAW resonator comprising the biosensor components described herein. Building a BAW sensor for liquid or gas sensing is based on the principle that anything that interacts directly with the surface of the BAW sensors will change its resonant frequency. By tracking and decoding the resonant frequency (measure or phase frequency), the mass loading and the viscosity of the particles attached to the surface of the sensor can be measured.

### Biocoating Method

Some embodiments relate to a process of coating a surface of a material with a bioactive film by applying a first composition comprising an anchor substance to the surface of the metal material to form a monolayer on the surface, wherein the anchor substance comprises a binding protein and a functional group having at least one sulfur and/or applying a second composition comprising a biotinylated capture reagent to the monolayer of the anchor substance, wherein the biotinylated capture reagent binds to the anchor substance through the binding protein to form a layer of the biotinylated capture reagent.

Some embodiments relate to a process of coating a metallic surface with a bioactive film by applying a first composition comprising an anchor substance to the aluminum, gold, silicone dioxide or PMMA surface to form a monolayer on the sensor surface, wherein the anchor substance comprises a binding protein and a thiol functional group and/or applying a second composition comprising a biotinylated capture reagent to the monolayer of the anchor substance, wherein the biotinylated capture reagent binds to the anchor substance through the binding protein to form a layer of the biotinylated capture reagent.

Some embodiments relate to a process for coating the surface of a piezoelectric material with biofilm comprising an anchor substance having the property of binding to a capture reagent comprising or constituting a specific binding partner for the anchor substance, the process comprises treating a substrate surface of a piezoelectric material to activate the substrate surface and applying a layer of the anchor substance directly to the activated surface of the piezoelectric substrate.

In some embodiments, the method comprises introducing a signal amplifying material having one or more recognition sites to bind an analyte.

Some embodiments provide a process for coating an aluminum surface with biofilm comprising an anchor substance having the property of binding to a capture reagent comprising or constituting a specific binding partner for the anchor substance, the process comprises applying a layer of the anchor substance to the treated aluminum surface to form an anchor layer on the piezoelectric surface, wherein the anchor substance comprises a thiol functional group.

Some embodiments provide a method for determining the presence or quantity of an analyte in a biological fluid sample the method comprises contacting the foregoing biosensor component with a composition comprising a capture reagent the capture reagent comprising or constituting a specific binding partner for the anchor substance and also specifically recognizing an analyte causing the capture reagent to bind to the anchor substance, forming a capture reagent layer, contacting the bound capture reagent layer with a biological fluid sample which causes the signal amplifying material to bind to the analyte and generates an acoustic wave across/through the piezoelectric surface and measures any change(s) in amplitude, phase, time-delay or frequency of the wave as a result of the analyte binding to the capture reagent layer.

In some embodiments, the method described herein further includes activating the surface of the anchor substance. In some embodiments, activing the surface of the anchor substance comprises plasma cleaning.

In some embodiments, the method described herein is a direct coating. In some embodiments, the coating involves simple and rapid coating chemistries that are executed in seconds or minutes rather than hours. The direct coating can be manufactured using a scalable, continuous and in-line method such as ink-jet printing, which requires precision and ability to dispose a monolayer of substance easily automated with minimal operator intervention. This produces a low number of rejects and generates smaller amounts of hazardous waste. This coating method deposits anchor substances directly on the piezoelectric surface without an intermediary layer of material.

In some embodiments, the preparation method described herein comprises cleaning of the piezoelectric substrate surface. The cleaning step can be accomplished by a number of methods, including but not limited to acid treatment, ultraviolet exposure and various methods of plasma treatment which can remove virtually all organic contaminants on the surface of the piezoelectric substrate via the generation of highly reactive species. In some embodiments, the preparation method comprises plasma cleaning.

In some embodiments, the method described herein further includes activating the surface of the anchor substance. In some embodiments, activing the surface of the anchor substance comprises plasma cleaning which includes using oxygen or oxygen/argon mixture to treat the surface. The plasma cleaning can last for 1-10 min, 1-20 min, 1-30 min, or 1-60 min. The plasma cleaning could last for longer than 1 min, 5 min, 10 min, 20 min, 30 min, 40 min, 50 min, 60 min, 1.5 h, 2 h, 3 h, or 4 h. In some embodiments, the plasma cleaning lasts for shorter than 5 min, 10 min, 20 min, 30 min, 40 min, 50 min, 60 min, 1.5 h, 2 h, 3 h, or 4 h. In some embodiments, the plasma cleaning includes treatment at 50-200 watts of 50-150 KHz.

Avidins are proteins derived from egg whites, e.g., from avian reptile and amphibian species, and have been used in many biochemical reactions. The avidin family includes neutravidin, streptavidin and avidin, all proteins functionally defined by their ability to bind biotin with high affinity and specificity. Avidins can also include bacterial avidins such as streptavidin and modified avidins like neutravidin (e.g., deglycosylated avidin from Thermo Scientific). They are small oligomeric proteins, each comprising four (or two) identical subunits, each subunit bearing a single binding site for biotin. When bound to the surface of the biosensor in the present disclosure, some sites may be facing the metal coated piezoelectric material surface, and are therefore unavailable for biotin binding. Some other sites are facing away from the piezoelectric material and are therefore available for biotin binding. The binding affinity of avidins to biotin, albeit noncovalent, is so high that it can be considered irreversible. The dissociation constant of avidin (KD) is approximately 10-15 M, making it one of the strongest known non-covalent bonds. In its tetrameric form, avidin is estimated to be between 66 to 69 kDa in size. Ten percent of the molecular weight is attributed to carbohydrate content composed of four to five mannose and three N-acetylglucosamine residues. The carbohydrate moieties of avidin contain at least three unique oligosaccharide structural types that are similar in structure and composition.

Biotin, also known as d-biotin or Vitamin H, Vitamin B7 and Coenzyme R, is a specific binding partner of avidin. It is commercially available from multiple suppliers, including Sigma-Aldrich.

Biosensor technologies described herein allow for the use of acoustic methods for biological sensing with high accuracy and sensitivity. The technologies described herein can be used to accommodate and bind biologically sensitive agents onto the surface of the acoustically transmissive materials, which helps further expand the use of acoustic methods for detection applications. Some embodiments relate to the use of a signal amplifying material to enhance the signal generated from the binding of the target analyte to the biosensor and increase the sensitivity of the biosensor by many folds.

The intrinsic sensitivity of SAW sensors can be high, and detection of a variety of biological analytes can be in the nanogram to picogram range or even, in some cases, in the femtogram range. However, the sensitivity of some SAW biosensors may be insufficient for the detection of ordinary biological analytes in the high picomolar range and also for the detection of bacterial or viral infections where the number of infectious particles in biological fluids may be small. Further, as the volumes of biological fluids are also limited, the detection methods with low sensitivity, are often not applicable.

The detection and quantifying methods described herein can have sensitivity sufficient to detect biological analytes in the high to low picomolar range. The techniques herein may also allow for the detection of bacterial or viral infections where the number of infectious particles in biological fluids are small (i.e., <10 particles/ml). In addition, the enhanced sensitivity of the detection method described herein can also be used when the volumes of biological fluids are also limited (e.g., 10-250 microliters).

### Methods of Amplifying Signal

Some embodiments relate to a method of amplifying a signal for biosensors. The methods may include (i) applying a sample to the biosensor having a capture reagent having one or more first recognition moieties for binding an analyte, and (ii) introducing a signal amplifying material to the biosensor. The capture reagent is immobilized on the biosensor via an anchor substance (e.g., avidin, antibodies, antibody fragments, polypeptides, and the like). When the sample is exposed to the biosensor, the analyte in the sample binds to the capture reagent on the surface of the biosensor. The signal amplifying material includes materials that can increase the mass of the surface bound moieties. Examples of the signal amplifying material may include biomolecules, polymeric material or metallic or metal oxide material. The signal amplifying material ideally also has one recognition moiety configured to bind the analyte that is not bound to the capture reagent on the biosensor.

The binding of the signal amplifying material adds mass to the surface bound analyte, and therefore, increases the amplitude of the biosensor signal and sensitivity. In some embodiments, the signal amplifying material is a biomolecule, polymer or metallic or metal oxide material. In some embodiments, the signal amplifying material can be in the form of particles. In some embodiments, the signal amplifying material can be a metallic material. In some embodiments, the signal amplifying material can be metal or metal oxide particles. In some embodiments, the metallic material can be Au, Pd, or Pt. In some embodiments, the signal amplifying material can be oxide material such as SiO2 and iron oxide, and quantum dot. In some embodiments, the signal amplifying material is a polymer particle. In some embodiments, the particle can be in the form of a bead made up of polymer or some such material with a defined size and mass. In some embodiments, the signal amplifying material is polystyrene. In some embodiments, the signal amplifying material is polystyrene with one or more fluorescent dyes. In some embodiments, the signal amplifying material can be melamine resins (MF), polystyrene (PS), polydivinylbenzene (PDVB), or polymethyl methacrylate (PMMA). In some embodiments, the signal magnifying material also includes one or more fluorescent dyes.

In some embodiments, the signal amplifying material is a particle in the form of a bead. In some embodiments, the particle has an average diameter in the range of about 1 nm to about 100 μm or about 10 nm-100 nm. In some embodiments, the particle has an average diameter in the range of about 10 nm-about 500 nm, about 100 nm-about 400 nm, about 100 nm-about 300 nm, about 100 nm-about 250 nm, about 150 nm-about 250 nm, about 170 nm-about 230 nm. In some embodiments, the particle has an average diameter of greater than about 1 nm, greater than about 5 nm, greater than about 10 nm, greater than about 50 nm, greater than about 75 nm, greater than about 100 nm, greater than about 150 nm, greater than about 200 nm, or greater than about 300 nm. In some embodiments, the particle has an average diameter of less than about less than about 250 nm, less than about 300 nm, less than about 400 nm, or less than about 500 nm.

In some embodiments, the polymer particle can have a mass of greater than about 1 femtogram (fg), greater than about 2 fg, greater than about 5 fg, greater than about 10 fg, greater than about 15 fg, greater than about 20 fg, greater than about 50 fg, greater than about 75 fg, greater than about 100 fg, or greater than about 200 fg. In some embodiments, the polymer particle can have a mass of less than about 5 fg, less than about 10 fg, less than about 50 fg, less than about 75 fg, less than about 100 fg, less than about 200 fg, or less than about 500 fg. In some embodiments, the polymer particle can have a mass in the range of 1 fg-about 20 fg, about 1 fg-about 100 fg, about 1 fg-about 500 fg, about 10 fg-about 100 fg, about 10 fg-about 500 fg, about 50 fg-about 250 fg, about 50 fg-about 800 fg, or about 100 fg-about 1 fg.

In some embodiments, the signal amplifying material is introduced after the analyte binds to the biosensor. In some embodiments, the signal amplifying material is introduced with the analyte during the measurement. In some embodiments, the amplifying material will be premixed with analyte and then applied to the surface of the sensor during the measurement.

In some embodiments, the method described herein comprises measuring a base level signal prior to applying the sample to the biosensor. In some embodiments, the method described herein comprises measuring a test level signal after the binding of the signal amplifying material to the analyte. In some embodiments, the method described herein comprises comparing the base level signal to the test level signal to determine the concentration of the analyte in the sample. In some embodiments, the method described herein comprises comparing the base level signal to the test level signal to determine the presence of the analyte in the sample.

In some embodiments, the method described herein comprises comparing the test level signal and the standard curve to determine the concentration of the analyte in the sample. The standard curve of a particular signal amplifying material can be created by measuring and plotting the frequency, phase shift or rate of change of the frequency or phase shift at a different number or concentration of the signal amplifying material.

In some embodiments, the first recognition moiety is a moiety for binding to whole cells, bacteria, eukaryotic cell, tumor cell, virus, fungus, parasite, spore, nucleic acid, peptide, or protein and small molecules. In some embodiments, the first recognition moiety can be selected from the group consisting of ligand, antibody (whole, fragment or single domain), affimer, and aptamer.

In some embodiments, the second recognition moiety is a moiety for binding to whole cells, bacteria, eukaryotic cell, tumor cell, virus, fungus, parasite, spore, nucleic acid, peptide or protein or small molecules. In some embodiments, the second recognition moiety can be selected from the group consisting of antibody, antibody fragment, single domain antibody, affirmer and aptamer. In some embodiments, the second recognition moiety is conjugated with a signal amplifying material (e.g., polymer, metal, or metal oxide material).

In some embodiments, the sample is an environmental or biological sample. In some embodiments, the biological sample is blood, serum, plasma, urine, nasal or vaginal swab, sputum fecal matter, tears, cerebrospinal fluid, pericardial fluid, intraocular fluid, cyst fluid, or saliva.

Some embodiments relate to a method for determining the presence or quantity of an analyte in a sample. The method includes applying a sample to the biosensor having a capture reagent having one or more first recognition sites for binding an analyte, wherein the capture reagent is immobilized on the biosensor, introducing a signal amplifying material, wherein the polymer or metallic or metal oxide coupled with affimer or antibody or ligand has one or more second recognition sites to bind the analyte; and measuring any change in amplitude, phase or frequency of biosensor signal as a result of analyte binding to the signal amplifying material.

The methods described herein can significantly enhance the detection sensitivity when compared with prior art methods lacking the disclosed signal amplifying material. In some embodiments, the method described herein can increase the sensitivity by at least about 2 fold, about 5 fold, about 10 fold, about 25 fold, about 50 fold, about 100 fold, about 200 fold, about 500 fold, about 800 fold, about 1000 fold. In some embodiments, the method described herein can increase the sensitivity by at least about 5%, about 25%, about 50%, about 75%, or about 90%. In some embodiments, the method described herein can increase the sensitivity in the range of about 5%-about 200%, about 5%-about 500%, about 50%-about 500%, about 50%-about 1000%.

The methods described herein can significantly enhance the detection accuracy when compared with methods without using the signal amplifying material. The method described herein can have a sensitivity level of as low as about 0.01 pg, about 1 pg, about 5 pg, about 10 pg, about 50 pg, about 100 pg. The method described herein can have a sensitivity level in the range of about 0.01 pg-about 500 pg, about 1 pg-about 500 pg, or about 10 pg-about 100 pg.

### Binding of Analytes to the Coated Biosensor

In some embodiments, the bound avidin on the piezoelectric substrate surface requires activation to bind analytes of interest. The activation includes a biotinylated binder such as an antibody, which is specific to an analyte antigen of interest. The antibody or other agent is biotinylated prior to its affixation to the avidin-coated chip. The antibody can bind to its analyte antigen before or after it is affixed to the avidin substrate. The analyte biotinylated antibody complex can be formed outside of the sensor and the complex can be contacted with the sensor, whereby the biotin on the antibody will bind to the avidin-coated chip. Which of the two methods is preferred is dependent upon the analyte and on the sample processing. Both methods are within the scope of the present invention. Analysis of the surface coating with a particular antibody bound to avidin on the chip surface resulted in a determination for depth of 6 to 9 nm, again using atomic force microscopy (AFM), demonstrating that antibody is indeed bound to the avidin layer.

Antigen-specific biotinylated capture reagents are applied to form a second layer consisting of bound and excess free biotinylated reagent in a non-drying medium also containing protein stabilizers known in the art such as, but not limited to, sucrose, trehalose, glycerol and the like. Many agents can be biotinylated, the most commonly used amongst them is biotinylated antibodies, specifically recognizing an analyte of interest. Protein capture reagents can be biotinylated chemically or enzymatically. Chemical biotinylation utilizes various known conjugation chemistries to yield nonspecific biotinylation of amines, carboxylates, sulfhydryls and carbohydrates. It is also understood that N-hydroxy succinimide (NHS)-coupling gives biotinylation of any primary amines in the protein. Enzymatic biotinylation results in biotinylation of a specific lysine within a certain sequence by a bacterial biotin ligase. Most chemical biotinylation reagents consist of a reactive group attached via a linker to the valeric acid side chain of biotin. Enzymatic biotinylation is most often carried out by linking the protein of interest at its N-terminus, C-terminus or at an internal loop to a 15 amino acid peptide, termed AviTag or Acceptor Peptide (AP) (using biotinylation techniques known to one of skill in the art).

Once bound, the capture reagent is briefly exposed to heated air to effect partial removal of water from the applied fluid forming a protective and stabilizing gel that will ensure long-term stability of bound proteinaceous binders like antibodies in a non-drying gel layer which allows essentially complete time-dependent formation of the second antigen-specific binder layer. These glass-like layers are optionally dehydrated for storage in the presence of desiccant pellets of silica or molecular sieves inside the pouch of the cartridge. The upper chamber of the cartridge is sealed to form a fluidic compartment. The cartridge with said chamber is then sealed inside a plastic storage pouch, preferably in a N2 atmosphere.

The binding between anchor substance (avidin) and biotinylated capture reagent may cause a second capture reagent layer to form on the chip. Prior to use, any residual unbound biotinylated capture reagent and other components in the protective gel layer can be readily removed by a simple flush with an assay buffer or even with the specimen fluid during the analytical procedure. These sensors have been demonstrated to detect antigens.

Biosensors described herein can be used to detect a variety of agents and biochemical markers when outfitted with the appropriate biofilm coating which contains a capture agent that specifically binds to the analyte of interest. Examples of the uses to which this integrated biosensor can be put include human and veterinary diagnostics. Analyte is defined as any substance that is or that is found in or generated by an infectious agent and that can be used in detection including without limitation an oligonucleotide, nucleic acid, protein, peptide, pathogen fragment, lysed pathogen, and antibody including IgA, IgG, IgM, IgE, enzyme, enzyme co-factor, enzyme inhibitor, toxin, membrane receptor, kinase, Protein A, Poly U, Poly A, Poly Lysine, polysaccharides, aptamers, and chelating agents. Detection of antigen-antibody interactions has been previously described (U.S. Pat. Nos. 4,236,893, 4,242,096, and 4,314,821, all of which are expressly incorporated herein by reference). Further, the application in the detection of whole cells (including prokaryotic), such as pathogenic bacteria and eukaryotic cells, (including mammalian tumor cells), viruses (including retroviruses, herpes viruses, adenoviruses, lentiviruses, etc.), fungus, parasites and spores, (included phenotypic variations, of infections agents, such as serovars or serotypes) are within the scope of the invention.

### Method of Preparing the Signal Amplifying Material

Antibody, antibody fragment and single domain antibody, affimer or aptamer can be physiosorbed on the surface of the signal amplifying material/particles or could be covalently conjugated on the surface of the signal amplifying material/particles. Functionalized polymer, metal or metal oxide material with COOH or NH2 or maleimide or Epoxy or neutravidin can be purchased. These particles can be covalently conjugated with antibody, antibody fragment and single domain antibody, affimer or aptamer using, for example, EDC/NHS or carbodiimide or maleimide chemistries. On the other hand, biotinylated antibody, antibody fragment and single domain antibody, affimer or aptamer can be used to couple the neutravidin/avidin conjugated particles. Affimer can also be conjugated with metal nanoparticles with any available SH-moiety.

### Example 1

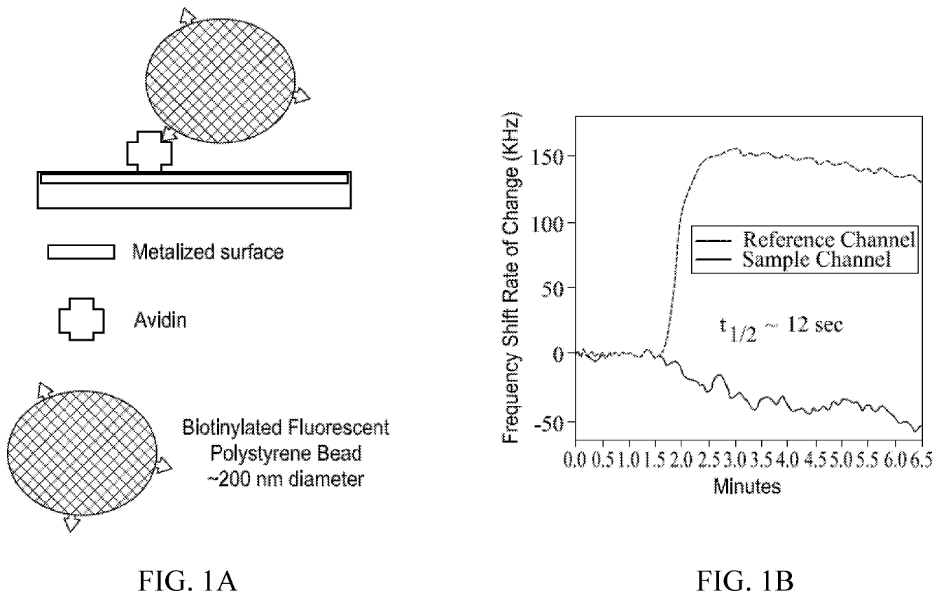

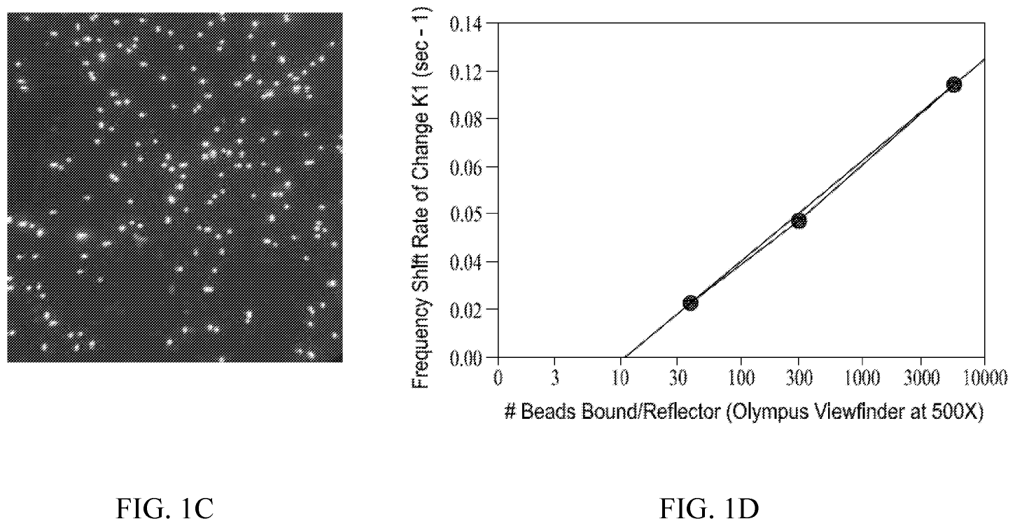

FIG. 1 shows that the SAW sensor used can readily detect a surface mass change in the low picogram range with indications of sensitivity in the mid to high femtogram range in a saline environment.

In FIG. 1A, the Aluminum or crystal surface of the SAW sensor was decorated with thiolated-neutravidin at a density of about one billion copies/mm2. In FIG. 1B, after washing away excess protein, frequency shifts of the sample (blue or top) and reference channels (red or bottom) were monitored. At 1.6 minutes, a 1:103 dilution of biotinylated fluorescent polyethylene beads (average diameter 200 nm) in saline was added to the sample channel (reference received saline only). Rapid binding of the biotinylated beads to the surface avidin (left panel) was detected by a frequency shift (center panel) whose half time (t ½) was about 12 s. No response was observed to saline in the reference channel. In FIG. 1C, following extensive and vigorous washing of the device, fluorescence microscopy confirmed tight binding of about 300 beads to the avidin decorated surface of the SAW sensor. In FIG. 1D, when similar experiments (not shown) were repeated with different bead dilutions, the absolute frequency shift as well as the rate of change (expressed as a rate constant, K1) of the frequency shift(s) were linearly related to the number of beads bound over three orders of magnitude. Assuming a linear extrapolation of the relationship to the x axis, the binding of about 11 beads appears to be the minimum threshold that can elicit an electrical response with the current non-optimized platform. The binding of 11 beads corresponded to a surface mass change of the sensor of 75 femtograms (i.e., 75×10-15 grams).

### Example 2

The following example and operating principle refers, but is not limited to, the enhanced detection of infectious agents with specific reference to the Zika virus but applies to and allows for the detection of even small molecules with enhanced sensitivity.

During the acute maximal phase of Zika and Dengue-related viremia, the maximal circulating concentrations of viral coat components useful for diagnosis are believed to span the picogram to nanogram/microliter range (Alcon et al). However, the circulating concentrations of the viral coat components (e.g., E protein) are at least two and possibly several orders of magnitude lower in both the very early and chronic stages of infection. Thus, a SAW based detection device ideally would require a working sensitivity in the very low femtogram range.

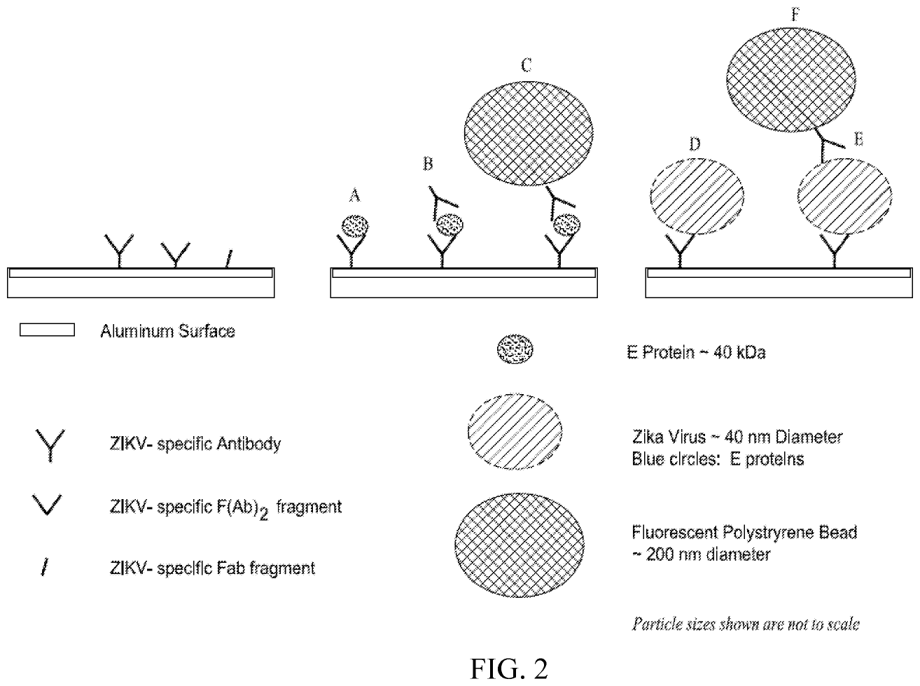

FIG. 2 illustrates a method that employs pairs of antibodies (or their Fab fragments, or aptamers, etc.) in a sandwich format to achieve enhanced level of sensitivity. As the SAW sensor is mass-sensitive, the addition of a second antibody after the desired analyte (blue dot) has already been captured by the surface bio-coating, adds additional mass to the sensor and thereby improves the sensitivity for any given analyte. When the second antibody is itself tagged with a very much larger mass (FIG. 2 green ball, e.g., a polystyrene or gold bead), the resultant increase in mass bound to the sensor can be many orders of magnitude greater than that of the original analyte or the second antibody itself.

The mass of a single 200 nm polystyrene bead (2.51 femtograms) can be nearly 5 orders of magnitude greater than that of an E protein monomer (0.066 attograms). Thus, the impact of the double amplification strategy on analyte sensitivity is massive. For example, simple calculations, based upon the preliminary data in FIG. 1, suggest that with a sandwich approach using double amplification (C in FIG. 2) the SAW sensor could detect less than one tenth of the E proteins arising from a single dissociated ZIKA virus (i.e., one tenth of a virus equivalent). In contrast, the same SAW sensor decorated only with one antibody (A and D in FIG. 2) would require the equivalent of about 200 ZIKA virions to achieve the same signal. Further, even larger diameter polystyrene beads (a 1 μm diameter bead has slightly more than 100× the mass of a 200 nm bead) can be used for additional increases in sensitivity. Moreover, polystyrene beads can be substituted with high density metallic beads (e.g., gold) to gain even further increases in sensitivity. Thus, the use of a sandwich technique enhanced by mass amplification is a novel means to dramatically augment the analyte sensitivity of any bio-coated SAW device. Moreover, mass amplification can be used with any analyte (large particles down to small molecules) for which specific pairs of antibodies or aptamers are available.

1. A method of amplifying signal in a biosensor, comprising:
applying a sample to the biosensor having a capture reagent, wherein the capture reagent comprises one or more first recognition moieties for binding an analyte, and wherein the capture reagent is immobilized on the biosensor; and
introducing a signal amplifying material, wherein the signal amplifying material has one or more second recognition moieties for binding to the analyte.

2. The method of claim 1, the signal amplifying material is a polymer, metal, or metal oxide.

3. The method of claim 1, wherein the signal amplifying material is polystyrene.

4. The method of claim 1, wherein the signal amplifying material is in the form of a bead of various materials.

5. The method of claim 4, wherein the bead has an average diameter in the range of about 1 nm to about 100 μm.

6. The method of claim 1, wherein the signal amplifying material is introduced after the analyte binds to the biosensor.

7. The method of claim 1, wherein the signal amplifying material is introduced prior to the analyte binding to the biosensor.

8. The method of claim 1, comprising measuring a base level signal prior to applying the sample to the biosensor.

9. The method of claim 1, comprising measuring a test level signal after the binding of the signal amplifying material to the analyte.

10. The method of claim 1, comprising comparing the base level signal to the test level signal to determine the presence of the analyte in the sample.

11. The method of claim 1, wherein the first recognition moiety is a moiety for binding to whole cells, bacteria, eukaryotic cell, tumor cell, virus, fungus, parasite, spore, nucleic acid, small molecule, or protein.

12. The method of claim 1, wherein the first recognition moiety is selected from the group consisting of antibody, antibody fragment, single domain antibody, affirmer and aptamer.

13. The method of claim 1, wherein the second recognition moiety is a moiety for binding to whole cells, bacteria, eukaryotic cell, tumor cell, virus, fungus, parasite, spore, nucleic acid, or protein.

14. The method of claim 1, wherein the second recognition moiety is selected from the group consisting of antibody, antibody fragment, single domain antibody, affirmer and aptamer conjugated with polymer, metal or metal oxide material.

15. The method of claim 1, wherein the sample is an environmental or biological sample.

16. The method of claim 15, wherein the biological sample is blood, serum, plasma, urine, sputum, fecal matter, nasal or vaginal swab, tears, cerebrospinal fluid, pericardial fluid, intraocular fluid, cyst fluid or saliva.

17. A method for determining the presence or quantity of an analyte in a sample the method comprising:
applying a sample to the biosensor having a capture reagent having one or more first recognition sites for binding an analyte, wherein the capture reagent is immobilized on the biosensor;
introducing a signal amplifying material, wherein the polymer or metallic material has one or more second recognition sites to bind the analyte; and
measuring any change in amplitude, phase or frequency of biosensor signal as a result of analyte binding to the signal amplifying material.

18. A biosensor component comprising: a piezoelectric substrate;
a capture reagent, wherein the capture reagent is immobilized on the piezoelectric substrate and wherein the capture agent has a first recognition site for an analyte, and
a signal amplifying material having a second recognition site for the analyte,
optionally further comprising an anchor substance to attach the capture reagent to the piezoelectric substrate.

19. (canceled)

20. The biosensor of claim 18, wherein the piezoelectric substrate is selected from the group consisting of aluminum (Al), lithium niobate (LiNbO3), lithium tantalate (LiTaO3), silicon dioxide (SiO2), and borosilicate.

21. The biosensor component of claim 20, wherein the anchor substance binds to the surface of the piezoelectric substrate through a silane group or a thiol group.

22. The biosensor component of claim 20, wherein the anchor substance comprises a linker protein selected from avidin, oligonucleotide, or polynucleotide, optionally wherein the linker protein is avidin selected from the group consisting of neutravidin, natural avidin, streptavidin, and any combination thereof.

23. (canceled)

24. The biosensor component of claim 18, wherein the capture reagent comprises a biotin moiety for binding to the linker protein of the anchor substance.

25. The biosensor component of claim 18, wherein the first recognition site is configured to bind whole cells, bacteria, eukaryotic cell, tumor cell, virus, fungus, parasite, spore, nucleic acid, or protein.

26. The biosensor component of claim 18, further comprising an acoustic wave transducer, wherein the acoustic wave transducer generates bulk acoustic waves (BAW) or surface acoustic waves (SAW).

27. (canceled)

28. The biosensor component of claim 26, wherein the BAW is selected from the group consisting of thickness shear mode, acoustic plate mode, and horizontal plate mode.

29. The biosensor component of claim 18, wherein the biosensor component is a film bulk acoustic-wave resonator-based (FBAR-based) device.

30. (canceled)

31. The biosensor component of claim 26, wherein the surface acoustic wave is selected from the group consisting of shear horizontal surface acoustic wave, surface traverse wave, Rayleigh wave, and Love wave.

32. A bulk wave resonator comprising the biosensor component of claim 18.
